(19) United States
(12) Patent Application Publication
(10) Pub. No.: US 2016/0331280 A1
(43) Pub. Date: Nov. 17, 2016

(54) A SYSTEM INTENDED FOR MEASURING, EVALUATING AND/OR GIVING FEEDBACK ON THE SITTING POSTURE OF A USER

(71) Applicant: NeuroMedicine Behavior Lab Scandinavia AB, Limhamn (SE)

(72) Inventor: Olof Arvid Huldt, Limhamn (SE)

(73) Assignee: NeuroMedicine Behavior Lab Scandinavia AB, Limhamn (SE)

(21) Appl. No.: 15/104,864

(22) PCT Filed: Dec. 11, 2014
(86) PCT No.: PCT/SE2014/051486

§ 371 Date: Jun. 15, 2016

(30) Foreign Application Priority Data
Dec. 19, 2013 (SE) 1351534-1

(51) Int. Cl. A61B 5/11; A61B 5/00

(52) U.S. Cl. CPC A61B 5/1116; A61B 5/1121; A61B 5/486; A61B 5/7405; A61B 5/7455; A61B 5/6823; A61B 5/6838; A61B 2562/0219

(57) ABSTRACT

The present invention describes a system intended for measuring, evaluating or giving feedback on the sitting posture of a user, said system comprising an upper body unit 1 comprising a battery, a microcontroller, an accelerometer and a feedback-unit, said upper body unit intended for measuring an angle of the thorax at an upper body position of the user; and a pelvis unit 2 comprising a battery, a microcontroller, an accelerometer and a feedback-unit, said pelvis unit intended for measuring an angle of the pelvis of the user.

### FIELD OF THE INVENTION

The present invention relates to a system intended for measuring, evaluating or giving feedback on the sitting posture of a user.

### TECHNICAL BACKGROUND

The number of people spending all their work day in front of a desk and a computer is vast and increasing. A lot of these people suffer from pain in the back, arm and neck. Some of these pains can be correlated to a poor sitting posture, which is widely accepted as a combination of a not suitable chair, shortage of abdominal muscle stamina, training and habituation. Common aids and treatments, usually delivered by physiotherapists, include lordosis pillows, new office chairs, height adjustable desks, foot supports, exercises for back and abdominal and leg-stretching walks at repeated intervals. In the field of sports, mainly golf, tennis, bowling (e.g. sports where twisting action is involved) there are a number of products measuring movements of spine, arms and thorax, to optimize performance and to prevent injury.

Furthermore, there are feedback systems directed to the sitting posture of a user. For instance, in US2008/0319351 there is disclosed a system and method for continually monitoring the spine (the lumbar region and cervical region of the spine) of a user via device containing electromechanical transducer, processor, delay (time switch), transmitter, and a feedback component. Through the device and method of employing, the user will be able to learn and sustain a proper posture, thereby avoiding future back pain.

Moreover, in US2013/0015976 a system and method are described for a sensor device which biomechanically detects in real-time a user's movement state and posture and then provides real-time feedback to the user based on the user's real-time posture. The feedback is provided through immediate sensory feedback through the sensor device (e.g., a sound or vibration) as well as through an avatar within an associated application with which the sensor device communicates. The postural feedback apparatus according to US2013/0015976 comprises a sensor device configured to be attached on a user, wherein the sensor device comprises a tri-axial accelerometer; an actuator; and a microprocessor configured to receive data from the tri-axial accelerometer about movement of the user; normalize the received accelerometer data; determine a postural description of the user based on the normalized received accelerometer data; and trigger the actuator to output sensory feedback based on the postural description of the user.

One problem with the systems disclosed above is the high complexity of the systems. Another problem is related to the comparatively low user-friendliness of these systems.

One purpose of the present invention is to provide a system which is effective and highly accurate. Moreover, another purpose of the present invention is to prove a system which is easy to use on a daily basis and which is beneficial for all type of users.

### SUMMARY OF THE INVENTION

The stated purposes above are achieved by a system intended for measuring, evaluating and/or giving feedback on the sitting posture of a user, said system comprising:

an upper body unit comprising a battery, a microcontroller, an accelerometer and a feedback-unit, said upper body unit intended for measuring an angle of the thorax of the user; and
a pelvis unit comprising a battery, a microcontroller, an accelerometer and a feedback-unit, said pelvis unit intended for measuring an angle of the pelvis of the user.

The present invention is directed to system involving the measurement in two very different locations, namely in one upper body (thorax) location and also in one pelvis location. This is different when comparing to the systems disclosed above. This feature according to the present invention also renders several advantages. First of all, this feature is the base for yielding a high accuracy and measurement stability of the evaluation tool. Secondly, it is also the base for providing a system which is easy for anyone to use and which is not interfering with the personal sphere of the user, and as such is non-stigmatizing. This is further disclosed below.

The two-point measurement according to the system of the present invention enables that the feedback to the user will be provided where it should be provided (at the site/location where the “problem” is) and also visualizes for the user which part of the body the user has to work with for improving the sitting posture. As such, the two-point measurement according to the present invention provides an increased spatial resolution and therefore increased accuracy of the feedback provided to the user when being compared with existing products and systems.

Furthermore, as understood from above, each unit of the system comprises a battery, a microcontroller, an accelerometer and a feedback-unit. Moreover, each unit may also comprise a user input function, such as a button or touch screen capability, for the user to be able to turn on/off the units, calibrate the system, etc.

In the article “Comparison of four specific dynamic office chairs with a conventional office chair: Impact upon muscle activation, physical activity and posture”, Applied Ergonomics, 2012, vol. 43, no. 2, pages 296-307, Ellegast R P et al, there is disclosed a system intended to evaluate the effects of four specific dynamic chairs on erector spinae and trapezius EMG, postures/joint angles and physical activity intensity (PAI) compared to those of a conventional standard office chair. From measured signals during analysis, the following body/joint angles and positions and their corresponding degrees of freedom were calculated: head: sagittal and lateral inclination; cervical spine: flexion/extension; thoracic spine: sagittal and lateral inclination; lumbar spine: sagittal and lateral inclination; thigh right/left: spatial position; and lower leg right/left: spatial position. Inclination angles were used as a measure for the valuation.

The present invention, however, relates to a system comprising an upper body unit comprising a battery, a microcontroller, an accelerometer and a feedback-unit, said upper body unit intended for measuring an angle of the thorax of the user; and a pelvis unit comprising a battery, a microcontroller, an accelerometer and a feedback-unit, said pelvis unit intended for measuring an angle of the pelvis of the user.

In the evaluation method disclosed in article “Comparison of four specific dynamic office chairs with a conventional office chair: Impact upon muscle activation, physical activity and posture”, Applied Ergonomics, 2012, vol. 43, no. 2, pages 296-307, Ellegast R P et al. many different body/joint angles and positions are used on the body of the person performing the test. There is no hint with reference to any specific points being of more interest in relation to the others. Furthermore, the method disclosed is an evaluation method and is not at all related to a system enabling to give feedback on the sitting posture of a user, such as according to the present invention. Therefore, the article gives no guidance with reference to suitable components of such a system. The present invention, however, relates to a system comprising components needed for being able to provide feedback to a user of a sitting posture.

### BRIEF DESCRIPTION OF THE DRAWINGS

FIG. 1a and FIG. 1b show a user utilizing a system according to the present invention, and where two different sitting postures are visualized.

FIG. 2 also shows a user utilizing a system according to the present invention, however in this case the user is walking.

FIGS. 3a and 3b show two possible flow chart working loops of the system according to the present invention, where FIG. 3a visualizes one possible way of calibrating the system and FIG. 3b shows one possible feedback mode of the system.

FIG. 4 shows a possible user and a sagittal plane thereof, said sagittal plane being a vertical plane, passing from ventral (front) to dorsal (rear), dividing the body of the user into right and left halves.

FIG. 5 shows a bra having an upper body unit attached thereto by means of said upper body unit having a clip-on configuration.

FIG. 6 shows a necklace holding an upper body unit.

FIG. 7 shows a belt according to one possible embodiment of the present invention, where the pelvis unit is attached to the belt.

FIG. 8 shows a clip-on configuration according to one embodiment of the present invention.

### SPECIFIC EMBODIMENTS OF THE INVENTION

Below, some specific embodiments of the present invention are disclosed. According to one embodiment, the feedback-units of the upper body unit and the pelvis unit are mechanical and/or audio directed. One possible mechanical feedback-unit according to the present invention is a vibration motor (vibration device). An audio directed feedback-unit is e.g. a device providing some kind of sound when feedback is provided. Such a property may for instance be of special interest for positive feedback, such as e.g. to be provided when a user goes back into a suitable sitting position from having been out of it for a while.

According to one specific embodiment of the present invention, the feedback-units of the upper body unit and the pelvis unit are mechanical and audio directed. In this case the feedback units are both mechanical and audio directed. One alternative is for the feedback units to have negative feedback system, such as e.g. provided by vibration, and one positive feedback system, e.g. with sound.

Also the accelerometers may have different functionality according to the present invention. First of all, uniaxial, biaxial and triaxial accelerometers are all possible, in duplicate or in different combinations. Moreover, also the measurement target of different axis of one accelerometer may vary. For instance, according to one specific embodiment of the present invention, one axis of each accelerometer is directed to measurement of an angular deviation from a horizontal axis as seen within a sagittal plane of the user, and one other axis of each accelerometer is directed to measurement of kinetic energy. When one axis is directed to the forward or backward position (direction nose—neck), a totally lateral movement of the user will not be detected by this axis. Furthermore, as a user is moving much up and down, the axis in that direction is very suitable for measuring kinetic energy, such as the accumulated kinetic energy created by the user. In the case of measuring kinetic energy, a gyroscope may be integrated and may register rotary motion.

The present system also has other advantages in comparison to existing systems and products used today. According to one specific embodiment of the present invention, the upper body unit and/or pelvis unit has means for attachment. As an example, the upper body unit may be fixated to a necklace or has means for attachment to a necklace, brooch or a bra and the pelvis unit has means for attachment at the hip of the user, such as for attachment to a belt or trousers or a skirt worn by the user. Accordingly, both the upper body unit and the pelvis unit may of course have means for attachment according to the present invention. According to one embodiment, the means for attachment has a clip-on configuration.

According to the present invention it is easy to wear the upper body unit and pelvis unit in an easy fashion and with a minimum risk of interfering with the personal sphere of the user. This provides user-friendliness and allows for people to wear and use the system very easy at work. Generally speaking, people in need of an aid for evaluating and giving feedback of the sitting posture, i.e. for training purposes, often are in the middle age or older, e.g. people not training enough and not keeping themselves in good enough shape. Other general user groups are persons who have had an accident and injured themselves and are in need of training. Regardless, if an aid is difficult or stigmatizing to use this barrier may be difficult to overcome for a user. For instance a tightly worn waist belt is one aid which in fact may be problematic for many possible users on a regular basis. Such a device is noticeable for the user all the times, and also something which is not easy for someone to use at work, e.g. in view of that it is very revealing of the shape of the body of the user, existing spare tires etc. As the present system is directed to a solution in which the units may be worn very easy and non-revealing, the barriers for using the system according to the present invention are very low.

The present invention is also directed to a method for measuring, evaluating and/or giving feedback on the sitting posture of a user. According to one specific embodiment, the present invention is directed to a method for measuring, evaluating and/or giving feedback on the sitting posture of a user, in which an upper body unit and pelvis unit in a system according to the present invention are attached to the user and switched on, wherein each microcontroller receive input data from respectively accelerometer and wherein the feedback-unit presents real-time output data and/or feedback to the user.

In relation to the present invention, the expression “real-time output data and/or feedback” implies, in its broadest sense, all kind of momentary output possible to present to the user. This implies that also mechanical feedback or audio-feedback should be encompassed by this expression. Real-time output data may be data in the form of actual measurements, such as e.g. of counting the numbers of going out from the range of a specific desired value of a parameter, specific time and actual values on a specific event, accumulated values, such as e.g. of the total kinetic energy, etc. etc. The data may of course comprise several different parameters and the feedback and data presented may e.g. include both plain feedback (e.g. mechanical or noise/sound) and data of some or several measurements.

According to one specific embodiment, the accelerometers are measuring at least one angle each and wherein the real-time output data is based on if said angle deviates from a set default/reference value or default/reference range. The normal/default value may be set by setting the “zero value” by using a calibration method. The calibration method according to the present invention is a simple procedure which may involve two steps: 1. Sitting in a posture determined and taught by a physiotherapist or other, to be a good position. 2. While in this position press or hold down the button(s) of the accelerometer units. There are ways of getting to know a good sitting posture (for some), for example by pointing towards the sky with a fully streched arm. As every user's need is somewhat uniqe it is difficult to set out one method for reaching a good sitting posture.

According to another specific embodiment, two or more units are used and their measured data is in combination used as a criteria, if feedback is to be given or not. That is, not only the simple deviation from a set value, but also a more complex combination of values, may be used as input.

According to yet another specific embodiment of the present invention, the accelerometers are measuring also at least the level of movement of the user over a certain time and wherein the feedback is based on if this level of movement deviates from a set default/reference value or default/reference range. The feedback unit may for instance also give feedback on the accumulated value of the level of movement.

Acceleration in a direction which reflects user movement is measured, for example the vertical direction is suitable for detecting walking as the head bounces up and down. Integration or a calculated mean of the absolute value of the accelerometer reading gives a measure of the users kinetic energy or amount of movement which is an input to the system if how and when to give feedback.

With reference to the embodiment disclosed above it should be said that the present invention may also be directed to giving positive feedback. As an example, positive feedback may be provided by the feedback unit if and when a user goes back into a suitable sitting posture when having been out from such for a while. The present invention may in fact utilize negative or positive feedback or both. Positive feedback may be of special interest when trying to change the behaviour of sitting and not only trying to evaluate it or measure important parameters.

The feedback unit may also comprise means for visualization. As an example, values of important parameters may be saved and after a full day's work, the feedback unit may visualize the sitting posture of the day for the user. This may provide information about possible problems with e.g. the chair used.

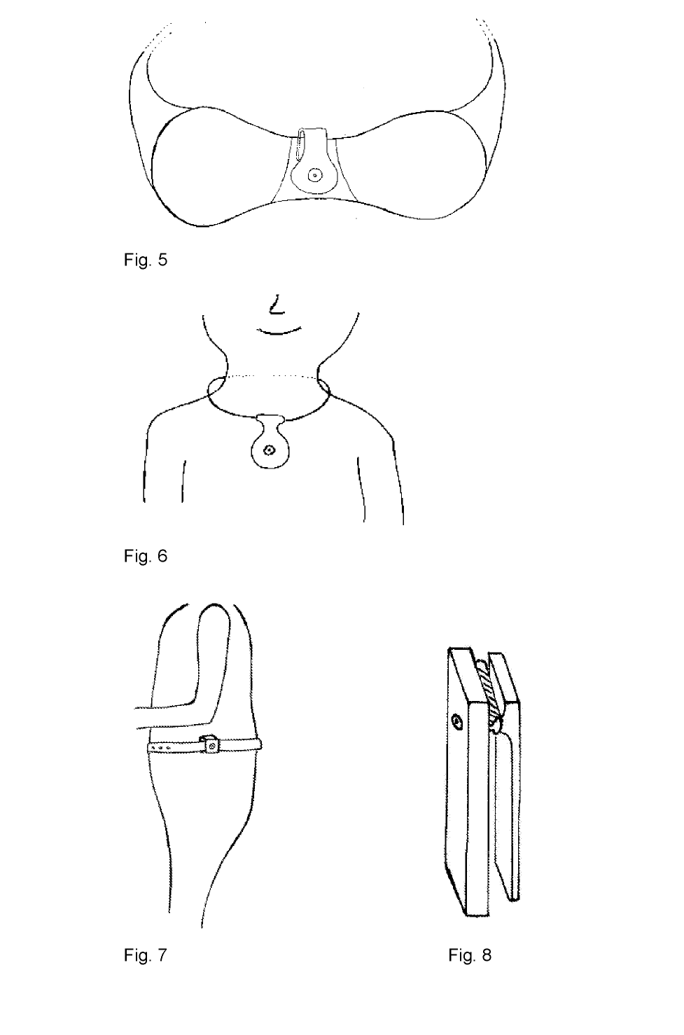

According to one embodiment of the method according to the present invention, the upper body unit is attached to a bra (see FIG. 5), attached to a necklace (see FIG. 6) worn by the user or positioned near the chest of the user, and wherein the pelvis unit is positioned at a hip (see FIG. 7) of the user. As mentioned above, this feature of the present invention provides a strong foundation for being a simple system to use on a daily basis, and which system is non-stigmatizing for the user. The system according to the present invention is e.g. not limited by specific clothing to be used, the weight and fitness of the user, etc. etc.

According to yet another specific embodiment there is disclosed the use of a system according to the present invention, for learning, training and/or improving a sitting posture of a user, for evaluating sitting means for a user and/or for logging sitting posture data of a user. The system according to the present invention may be used for diagnostic reasons. In such a case, the system may be connected to a logging device. The physiotherapist or doctor may use the system for diagnosing if e.g. pain is related to an incorrect sitting posture or not.

### DETAILED DESCRIPTION OF THE DRAWINGS

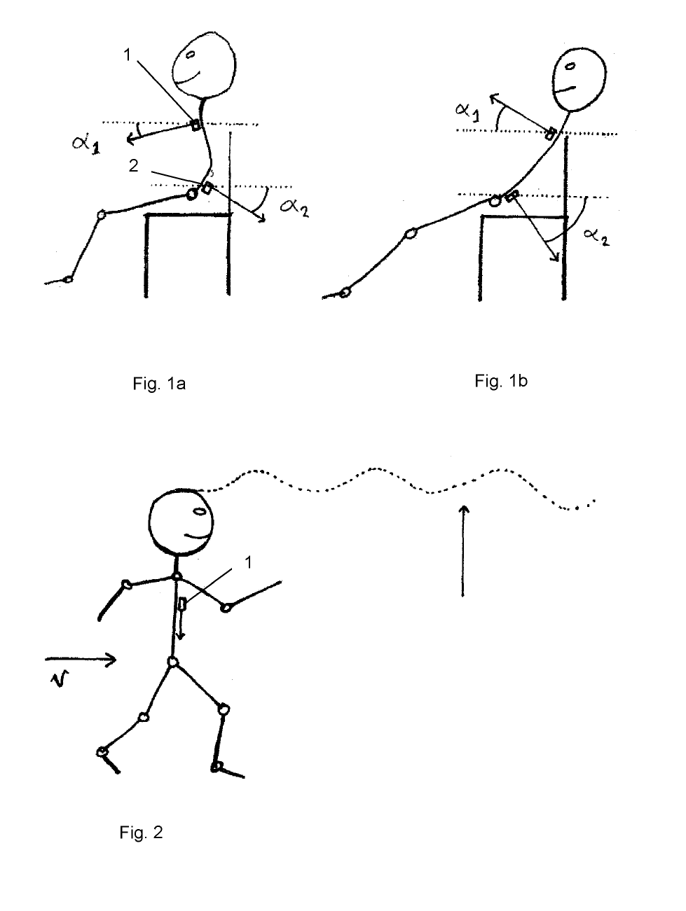

FIG. 1a and 1b show a user utilizing a system according to the present invention, said system comprising an upper body unit 1 and a pelvis unit 2, both having batteries, microcontrollers, accelerometers and feedback-units. It should be noted that both the upper body unit 1 and a pelvis unit 2 may be provided in multiple units in the system according to the present invention, all operating separately or in connection with each other, such as connected by wire or by some means of wireless communication. As seen, the units are registering angles (α₁ and α₂) within the sagittal plane (see the dotted lines) by measuring the portion of gravitational static acceleration in specific accelerometer axis. The angle within the sagittal plane are computed by simple trigonometry. The size of the static acceleration gives the angle in relation to the vertical plane. (1 g=straight down, 0 g=horizontal and −1 g=straight up).

The upper body unit 1 and a pelvis unit 2 are designed so that they may be in close contact with its measuring surface. Furthermore, and as seen, the upper body unit 1 is positioned on/at the thorax of the user, suitably in a necklace or fixated on the bra. The pelvis unit 2 may e.g. be fixated to the belt or trousers of the user.

As seen in FIG. 1a and 1b, the system according to the present invention provides two-point measurement (thorax and pelvis). This has several advantages, and these are summarized below together with other advantages of the system of the present invention:

A good sitting posture may be derived from holding both pelvis and thorax in certain angles, and this system is focusing on both and provides feedback at the correct places. As such the system may also provide feedback to the user regarding potential problematic regions of the sitting posture of a user;

Two point measurement provides higher spatial resolution and therefore increased accuracy of the feedback provided to the user;

Both units are affecting the personal sphere of the user very little. As such, the system is non-stigmatizing and provides high rate of use probability.

The system is easy to use on a regular basis;

It may be of importance to focus on the entire thorax and not only the angle at one or some vertebrae;

Focus on the thorax provides several suitable positions for fixation of the upper body unit (e.g. necklace, bra or brooch);

The front of the thorax, e.g. the chest, is not a fat collecting part of the body, and as such standardized (positive as a measurement point);

The pelvis angle is of importance in itself as it is possible to hold the thorax in a correct angle without having a suitable sitting posture; Fact is that this type of sitting posture probably causes most of the general incorrect sitting postures, which is caused by not having enough strength in the abdomen, back and pelvis;

The pelvis unit may also be fixated very easily to a belt or to trousers or underwear, e.g. by a clip-on (see FIGS. 7 an 8);

FIG. 2 also shows a user utilizing a system according to the present invention, however in this case the user is walking. The head of the user will move also in vertical direction (see the right and vertically directed arrow) which implies that an upper body unit accelerometer may give reading also in this direction. This may be utilized to measure kinetic energy.

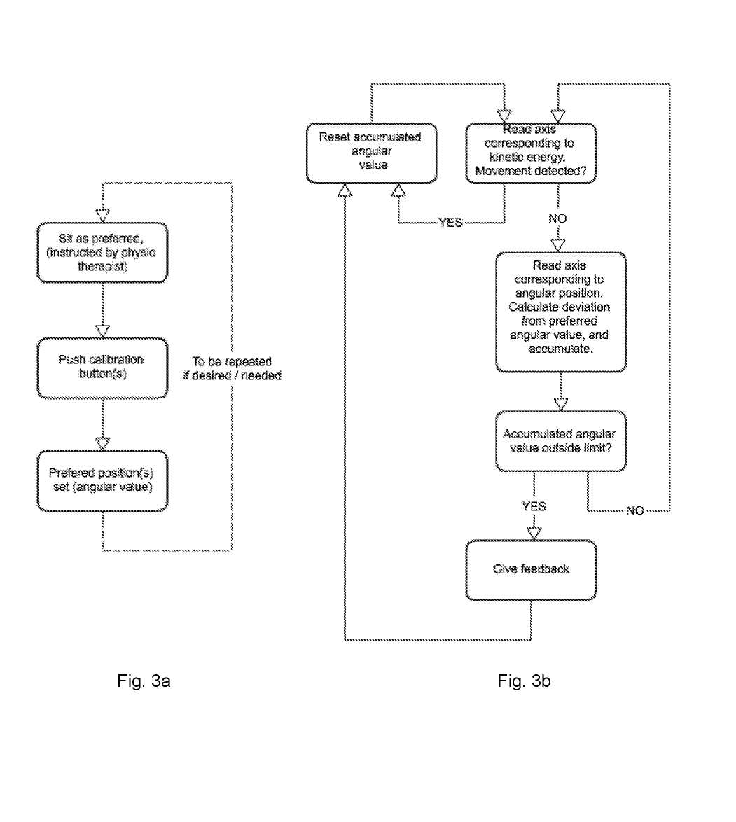

FIG. 3a shows one possible way of calibrating a system according to the present invention. The calibration according to the present invention may be directed to a zeroing procedure, such as e.g. a “tare” solution normally used for a weighing machine. The user sits in correct position, either by help of training, a routine or physiotherapist, for instance looks up in the ceiling whereby the back stretches, and then e.g. pushes a calibration button one second or so. As such, the preferred values or range values are set.

FIG. 3b shows one possible flowchart feedback mode of a system according to one embodiment of the present invention. As notable in this case, at least one axis of the accelerometer of the upper body unit detects and reads kinetic energy. If no movement is detected, then the other axis, e.g. on both accelerometers of the upper body unit and pelvis unit, reads the angles being directed to angular position. The deviation from a preferred value is calculated and e.g. the accumulated deviation value is calculated. If the measured real value of the angle deviates from the set desired value (or range) outside of a decided limit then feedback/stimulus is provided to the user so that the problem and incorrect angle may be corrected. The normal position is determined by a simple tare/zeroing or calibration procedure, e.g. like the one shown in FIG. 3a. Just as an example, possible feedback situations in a system according to the present invention are e.g. directed to deviation of the angular position, accumulated momentum, e.g. if a user has remained seated too long, positive feedback if a user goes back into a suitable position after being out from it during a certain time, all of which may create stimuli (such as mechanical feedback or audio-feedback), and e.g. more general feedback on data which may be visualized for the user, such as accumulated values and/or for example an indication or visualization of the sitting posture during a certain time.

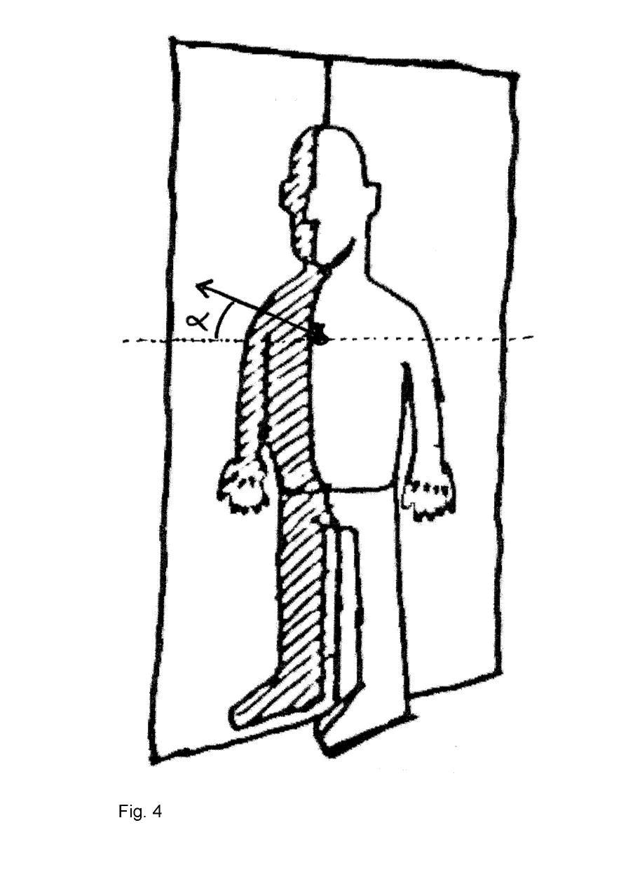

In FIG. 4 there is shown a sagittal plane of possible user. From this figure it is clear what the expression “angular deviation from a horizontal axis as seen within a sagittal plane of the user” is related to.

As mentioned above, FIG. 5-7 show different possible configurations according to the present invention. FIG. 5 shows a bra having an upper body unit having a clip-on configuration, FIG. 6 shows a necklace holding an upper body unit and FIG. 7 shows a belt having a pelvis unit attached thereto. Moreover, in FIG. 8 there is shown a clip-on configuration according to one embodiment of the present invention.

1. A system intended for measuring, evaluating and/or giving feedback on the sitting posture of a user, said system comprising:
an upper body unit comprising a battery, a microcontroller, an accelerometer and a feedback-unit, said upper body unit intended for measuring an angle of the thorax of the user; and
a pelvis unit comprising a battery, a microcontroller, an accelerometer and a feedback-unit, said pelvis unit intended for measuring an angle of the pelvis of the user.

2. The system according to claim 1, wherein the feedback-units of the upper body unit and the pelvis unit are mechanical or audio directed.

3. The system according to claim 2, wherein the feedback-units of the upper body unit and the pelvis unit are mechanical and audio directed.

4. The system according to claim 1, wherein one axis of each accelerometer is directed to measurement of an angular deviation from a horizontal axis as seen within a sagittal plane of the user and wherein one other axis of each accelerometer is directed to measurement of kinetic energy.

5. The system according to claim 1, wherein the upper body unit and pelvis unit has means for attachment.

6. The system according to claim 5, wherein the means for attachment has a clip-on configuration.

7. A method for measuring, evaluating and/or giving feedback on the sitting posture of a user, said method involving using a system comprising:
an upper body unit comprising a battery, a microcontroller, an accelerometer and a feedback-unit, said upper body unit intended for measuring an angle of the thorax of the user; and
a pelvis unit comprising a battery, a microcontroller, an accelerometer and a feedback-unit, said pelvis unit intended for measuring an angle of the pelvis of the user, in which method the upper body unit and pelvis unit in the system are attached to the user and switched on, wherein each microcontroller receive input data from respectively accelerometer and wherein the feedback-unit presents real-time output data and/or feedback to the user.

8. The method according to claim 7, wherein the accelerometers are measuring at least one angle each and wherein the real-time output data is based on if said angle deviates from a set default/reference value or default/reference range.

9. The method according to claim 7, wherein the accelerometers are measuring at least the level of movement of the user over a certain time and wherein the feedback is based on if this level of movement deviates from a set default/reference value or default/reference range.

10. The method according to claim 7, wherein the upper body unit is attached to a bra, attached to a necklace or brooch worn by the user or positioned near the chest of the user, and wherein the pelvis unit is a positioned at a hip of the user.

11. The method according to claim 7, said method involving using the system for learning, training and/or improving a sitting posture of a user, for evaluating sitting means for a user and/or for logging sitting posture data of a user.
